(19) United States
(12) Patent Application Publication
(10) Pub. No.: US 2010/0004495 A1
(43) Pub. Date: Jan. 7, 2010

(54) PROCESS FOR PRODUCING CARBON DIOXIDE AND METHANE BY CATALYTIC GAS REACTION

(76) Inventors: Erik Fareid, Langesund (NO); Marc Lambert, Langhus (NO); Tommy Scherning, Ski (NO)

Correspondence Address: FROMMER LAWRENCE & HAUG, 745 FIFTH AVENUE- 10TH FL., NEW YORK, NY, 10151, US

(21) Appl. No.: 12/447,359

(22) PCT Filed: Nov. 2, 2007
(86) PCT No.: PCT/NO2007/000387

§ 371 Date: Apr. 27, 2009

(30) Foreign Application Priority Data
Nov. 2, 2006 (NO) 20065021
Jun. 18, 2007 (NO) 20073080

(51) Int. Cl. C07C 1/04

(52) U.S. Cl. 585/310

(57) ABSTRACT

It is disclosed a process for producing methane and oxygen through the combustion of organic material, in said combustion there being formed carbon dioxide and carbon monoxide. The reaction is performed in a catalytic gas reactor in the presence of water.

### DISCLOSURE

With today's focus on human-produced CO₂ and the effect this substance has on pollution and global heating, it is of great importance to reduce or re-use and recirculate CO₂.

It is previously known different materials and methods for methanation and production of hydrogen. Examples of such prior art is represented by the following publications:
Jianjun Guo, Hui Lou, Hong Zhao, Dingfeng Chai and Xiaoming Zheng: “Dry reforming of methane over nickel catalysts supported on magnesium aluminate spines” Applied Catalysis A: General, Volume 273, no. 1-2, 8. October 2004, page 75-82;
M. Wisniewski, A. Boréave and P. Gélin: “Catalytic CO₂ reforming of methane over Ir/Ce_{0.9}Gd_{0.1}O₂₋ₓ,” Catalysis Communications, Volume 6, nbo. 9, September 2005, page 596-600;
Masaya Matsouka, Masaaki Kitano, Masato Takeuchi, Koichiro Tsujimaru, Masakazu Anpo and John M. Thomas: “Photocatalysis for new energy production. Recent advances in photo catalytic water splitting reactions for hydrogen production” Catalysis Today, 6. March 2007;
U. (Balu) Balachandran, T. H. Lee and S. E. Dorris: “Hydrogen production by water dissociation using mixed conducting dense ceramic membranes” International Journal of Hydrogen Energy, Volume 32, no. 4, March 2007, page 451-456;
Daniel M. Ginosar, Lucia M. Petkovic, Anne W. Glenn and Kyle C. Burch: “Stability of supported platinum sulfuric acid decomposition catalysts for use in thermo chemical water splitting cycles” International Journal of Hydrogen Energy, Volume 32, no. 4, March 2007, page 482-488;
T. Sano, M. Kojima, N. Hasegawa, M. Tsuji and Y. Tamaura: “Thermo chemical water-splitting by a carbon-bearing Ni(II) ferrite at 300° C.” International Journal of Hydrogen Energy, Volume 21, no. 9, September 1996, page 781-787;
S. K. Mohapatra, M. Misra, V. K. Mahjan and K. S. Raja: “A novel method for the synthesis of titania nano tubes using sono electro chemical method and its application for photo electro chemical splitting of water” Journal of Catalysis, Volume 246, no. 2, 10. March 2007, page 362-369;
S. K. Mohapatra, M. Misra, V. K. Mahajan and K. S. Raja: “A novel method for the synthesis of titania nano tubes using sono electro chemical method and its application for photo electro chemical splitting of water” Journal of Catalysis, Volume 246, no. 2, 10. March 2007, page 362-369;
Meng Ni, Michael K. H. Leung, Dennis Y. C. Leung and K. Sumathy: “A review and recent developments in photo-catalytic water-splitting using TiO₂ for hydrogen production”, Renewable and Sustainable Energy Reviews, Volume 11, no. 3, April 2007, page 401-425;
Wenfeng Shangguan: “Hydrogen evolution from water splitting on nano composite photo-catalysts” Science and Technology of Advanced Materials, Volume 8, no. 1-2, January-March 2007, page 76-81, APNF International Symposium on Nanotechnology in Environmental Protection and Pollution (ISNEPP2006);
Seng Sing Tan, Linda Zou and Eric Hu: “Photosynthesis of hydrogen and methane as key components for clean energy system” Science and Technology of Advanced Materials, Volume 8, no. 1-2, January-March 2007, page 89-92, APNF International Symposium on Nanotechnology in Environmental Protection and Pollution (ISNEPP2006);
U.S. Pat. No. 7,087,651 (Lee. Tuffnell et al., 8 Aug. 2006) “Process and apparatus for steam-methane reforming”;
U.S. Pat. No. 6,972,119 (Taguchi et al., Dec. 6, 2005) “Apparatus for forming hydrogen”;
U.S. Pat. No. 6,958,136 (Chandran et al., Oct. 25, 2005) “Process for the treatment of waste streams”;
U.S. Pat. No. 6,838,071 (Olsvik et al., Jan. 4, 2005) “Process for preparing a H₂-rich gas and a CO₂-rich gas at high pressure”.

The present invention may be summarized as a catalytic gas reactor including a catalyzer or process creating hydrogen and oxygen by splitting of water and a process with catalyzer creating methane from reactions wherein CO, CO₂ and hydrogen participate according to a methanation reaction scheme as follows:

CO+H₂O=CO₂+H₂ 1.

CO+3H₂=CH₄+H₂O 2.

CO₂+4H₂=CH₄+2H₂O 3.

H₂O=H₂+½O₂ 5.

The water is split into hydrogen and oxygen according to reaction 5 with several different processes. Some of these may be:
electrolysis of water at normal temperature,
water-splitting at high temperature over 2000° C.,
production of water from Ca—Br-cycle,
thermo chemical iodine-sulfur process at normal temperature,
ceramic membrane process at 200-900° C. (thermo chemical),
photo catalytic water-splitting with TiO₂,
photo catalysis with nano composite and catalyst consisting of cadmium sulphide (CdS) insert composite consisting of K₄Ce₂M₁₀O₃₀ (M=Ta,Nb) carrier coated with Pt, RuO₂ and NiO as contributing catalysts,
the creation of methane and hydrogen by photo catalysis by the use of TiO₂ catalyst,
all other systems creating hydrogen and oxygen from splitting of water and a combination thereof.

The methanation reaction may be performed with the catalysts infra with different compositions depending on the condition of the gas that is to be treated, but all methanation catalysts may be used in the temperature interval 150 to 600° C.;
Ni/NiO (nickel/nickel oxide) catalyst
Ru (ruthenium) catalyst
Cu (copper) catalyst
Pt (platinum)
Rh (rhodium)
Ag (silver)
Co (cobalt)
W (tungsten)
All other catalysts alone or together with one or more of the metals mentioned supra.

The advantage of the present invention is that CO₂ is transformed to methane through the aid of hydrogen and may consequently be used again as a fuel or as a raw material for a number of other processes. Some of these processes may be the production of methane, methanol, ammonia, urea, nitrous acid, ammonium nitrate, NPK, PVC, etc.

The present invention may be used in all forms of exhaust gases wherein fossil or biological fuel is used.

In addition the structure and composition of the reactors and catalyzers according to the present invention solves the problem with emission of VOC (volatile organic compounds), NOx (nitrogen oxides), N₂O (laughing gas), NH₃ (ammonia) and other greenhouse and in other ways polluting gases.

The present invention produces also energy far more effectively than similar processes today, and has far lower CO₂ emission per kWh than contemporary processes with CO₂ harvesting. Other advantages of the present process versus others are apparent from table 1 infra.

TABLE 1
Comparison between the present invention and similar
power plants withand without CO₂ collection. All
numbers* are relative to today's without CO₂ collection:

| | Contemporary | Contemporary | |
|---|---|---|---|
| | without CO₂- | with CO₂- | The present |
| | collection | collection | invention |
| Investment | 100 | 225 | 150 |
| CO₂-emission | 100 | 15 | 10 |
| Fuel consumption | 100 | 120 | 10 |
| Fuel cost NOK/h | 1200 | 1200 | 1200 |
| CO₂ tax NOK/h | 300 | 300 | 300 |
| CO₂ tax NOK/kWh | 0.16 | 0.024 | 0.013 |
| Fuel cost | 0.24 | 0.29 | 0.024 |
| NOK/kWh | | | |
| Financial cost | 0.09 | 0.21 | 0.13 |
| NOK/kWh | | | |
| Totoal cost | 0.49 | 0.52 | 0.17 |
| NOK/kWh | | | |

*All numbers are guiding

As a consequence of the development of the present invention, and as a non-separable part thereof, the present invention may be used within the general area of CO₂ purification, collection and sequestering.

The present invention is expressed as a reactor concept providing the industrial way of controlling the physical and chemical parameters involved in the following reaction equations:

CO+H₂O=CO₂+H₂ Shift reaction 1.

CO+3H₂=CH₄+H₂O Methanation reaction 2.

CO₂+4H₂=CH₄+2H₂O Methanation reaction 3.

CO₂+H₂=CO+H₂O Reverse shift reaction 4.

H₂O=H₂+½O₂ Water splitting 5.

The present reactions are also disclosed as the application of specific reactor designs providing catalytic and physical characteristics allowing and emphasizing the hydrogenation of CO₂ to CH₄ (methane).

The present invention may be considered as a dual one, the one part producing hydrogen and oxygen according to reaction 5. The other part will take advantage of the produced hydrogen from the first part, but may also individually produce hydrogen from reaction 1. The produced hydrogen will react with CO and CO₂ according to reaction 2 and 3 and produce methane. The produced methane and oxygen may either be re-circulated and combusted in a continuous loop or the methane and oxygen may be separated out and be used as a raw material for producing other chemicals.

Part 1 of the present invention may contain catalysts and other devices making it possible to use both the produced hydrogen and the produced oxygen.

Part 2 of the present invention is to contain a catalyst being suited for performing the methanation reaction, reactions 2 and 3, and suppressing the reverse shift reaction, reaction 4.

Part 1 and part 2 may be integrated with each other or may be separate entities.

Part 1 is the section wherein the water splitting is performed. This water dissociation needs much energy to happen. This energy may be taken from part 2 developing large amounts of energy or the energy may be provided from external sources.

The water may be split into hydrogen and oxygen according to reaction 5 through several different processes. Some of these may be:
electrolysis of water at normal temperature,
water dissociation at high temperature above 2000° C.,
production of water from Ca—Br cycle,
thermo chemical iodine-sulfur process at normal temperature,
ceramic membrane process at 300-900° C.,
photo catalytic water splitting with TiO₂,
photo catalysis with nano composite and catalyzers comprising cadmium sulphide (CdS) inclusion composite comprising K₄Ce₂M₁₀O₃₀ M=Ta, Nb) carrier coated with Pt, RuO₂ and NiO as contributing catalyzers,
production of methane and hydrogen by photo catlysis with the use TiO₂ catalysts,
All other systems creating hydrogen and oxygen from the dissociation of water,
dissociation may be performed with one of the systems or with two or more simultaneously.

In Part 2 the transforming of CO₂ with hydrogen to methane is performed in a reactor with a catalyst. The heat being developed may be used for heating part 1 or in any other way. The shape of the catalyst is not essential and may inter alia comprise coated monoliths, different nano materials and other types and forms of carriers. The carriers may be selected from e.g. TiO₂, Al₂O₃, cordierite, Gd-doped CeO and other types of carrier materials. The catalytic material may also be present in any form as a “pure” catalyst material. The form and composition of the reactor and the catalyst will depend on which emission gas it is wanted to purify. An impure exhaust gas with large amounts of dust (from the combustion of coal) may have a monolithic catalyst carrier whereas a pure exhaust gas (from a natural gas turbine) may have a catalyst in the form of pellets. All types of exhaust gases from all types of combustions of organic material may be treated.

The methanation reaction may be performed with the catalyzers infra with different compositions depending on the condition of the gas that is to be treated, but all methanation catalyzers may be used in the temperature interval 200 to 600° C.:
Ni/NiO (nickel/nickel oxide) catalyst
Ru (ruthenium) catalysts
Cu (copper) catalysts
Pt (platinum)
Rh (rhodium)
Ag (silver)
Co (cobolt)
W (tungsten)
All other catalysts alone or together with one or more of the metals mentioned supra.

When re-circulating the methane for further combustion and production of electricity or other forms of energy, the oxygen having been produced at the splitting of water may be used as a source for oxygen for the combustion of methane. Since air is not used as a source for oxygen, nitrogen will not participate as a diluting and reacting gas. Instead of nitrogen as a diluting gas (inert gas), water and CO₂ being produced at the combustion may be used. This gas (CO₂ and water) will be taken out for recirculation prior to the reactors having been disclosed in the present invention, and thus keeps a combustion temperature being commensurate with the materials that are present today for the construction of such combustion plants.

Nitrogen is the source for NOx at the combustion, and by performing the suggested recirculation the nitrogen will be replaced by CO₂ and water thereby avoiding the production of NOx. In avoiding NOx it is also possible to avoid the use of reducing measures creating laughing gas (N₂O).

Another theoretical solution for the use of the formed methane may be to produce methanol. This production may conceivably happen according to commercial processes being available today, and the methanol may have several areas of use such as e.g. fuel for transport means.

This process may conceivably be solved in the following way: Fuel is combusted with air in a burner. Electricity, optionally another form of energy, is taken out from the combustion process in the usual way. The CO₂ produced is used, as disclosed in the present invention, for producing methane. The methane is separated from the other gases and is used for producing methanol.

The present invention is not limited to these two fields, but may be used in all processes wherein natural gas or other hydrocarbons and organic compounds is one of the raw materials.

The present invention also produces energy far more efficiently than comparable processes today, and has a far lower CO₂ emission per kWh than today's processes with capture of CO₂. The other advantages of the present process as compare to others are observed in table 1 infra.

TABLE 1
Comparison between the present invention and comparable power
plants with and without capture of CO₂. All numbers a relative
to today's without capture of CO₂:

| | Present without | Present with | |
|---|---|---|---|
| | capture of CO₂ | capture of CO₂ | Present invention |
| Investment | 100 | 225 | 150 |
| CO₂ emission | 100 | 15 | 10 |
| Fuel consumption | 100 | 120 | 10 |
| Fuel cost NOK/h | 1200 | 1200 | 1200 |
| CO₂ tax NOK/h | 300 | 300 | 300 |
| CO₂ tax NOK/kWh | 0.16 | 0.024 | 0.013 |
| Fuel cost | 0.24 | 0.29 | 0.024 |
| NOK/kWh | | | |
| Financial cost | 0.09 | 0.21 | 0.13 |
| NOK/kWh | | | |
| Total cost | 0.49 | 0.52 | 0.17 |
| NOK/kWh | | | |

*All numbers are guiding

A small part of the exhaust gas must be emitted to avoid accumulation of certain trace elements. This exhaust gas contains mainly of CO₂ and water. This composition makes it very simple to capture CO₂ without using chemicals (e.g. amines and others), since the water may be condensed out while the CO₂ still is in a gaseous state. CO₂ may then be used for other purposes or may be stored. The cost for capture and optionally storage then become very small.

The disclosed reactions are common reactions (equilibrium reactions) happening in the production of ammonia over different catalytic layers.

The shift reaction happens in the LT or HT shift reactor wherein carbon monoxide reacts to produce carbon dioxide and hydrogen over a iron oxide/chromium oxide respectively a copper oxide/zinc oxide catalyst.

The methanation reaction happens in the methane reactor wherein carbon monoxide and carbon dioxide is reacted into methane and water over a nickel, ruthenium, tungsten or other metal-containing catalyst according to several total reactions (equilibrium reactions), inter alia:

CO+H₂O=CO₂+H₂ 1

CO+3H₂CH₄+H₂O 2.

CO₂+4H₂=CH₄+2H₂O 3.

Since the ammonia process is a process for producing ammonia via hydrogen from methane and nitrogen from air, the reactions 2. and 3. disclosed supra are reactions that are not wanted and which give losses of in the production of ammonia.

In the present invention all of these reactions are wanted since they produce methane being a product or intermediates participating in producing methane, and this effect has not previously been disclosed in the patent literature.

The source of carbon dioxide may be all kinds of combustion of organic materials such as emission gases or combustion gases from power plants, boats, cars, industrial plants that also include other contaminants. These contaminants may be, but are not limited to N₂O, NO, NO₂, volatile compounds (VOCs), SO₂, etc.

Ordinary destruction of these contaminants happens with CO₂ present in the combustion gas. An ordinary concentration of CO₂ in the combustion gas is about 1-20% by volume. When CO₂ is removed prior to the other contaminants the catalyst volume and the addition of chemicals will be reduced dramatically, partly on account of the lowered volume, and partly on account of the inhibitor effect of CO₂ if this is present.

Any process solution may be used for removing these contaminants.

The invention may be summarized by the following items:

1. A catalytic gas reactor including a catalyst and a process producing hydrogen and oxygen by dissociating water and a process with a catalyst producing methane from reactions wherein CO, CO₂, water, oxygen and hydrogen participate according to a methanation reaction scheme as follows:

CO+H₂O=CO₂+H₂ 1.

CO+3H₂=CH₄+H₂O 2.

CO₂+4H₂=CH₄+2H₂O 3.

H₂O=H₂+½O₂ 4.

### GENERAL USE OF THE INVENTION

The embodiments of the reactor are directed both towards new uses and reconstruction of existing devices for industrial combustion, and the invention of these rebuilding applications and new installations are claimed.

### BRIEF ACCOUNT OF THE FIGURES

FIG. 1: Catalytic CO₂ recirculation (CCR) technology;

FIG. 2: CCR technology with CO₂ recirculation (e.g. gas turbine or gas engine);

FIG. 3: CCR technology with CO₂ recirculation (e.g. with coal-fueled power plant);

FIG. 4: CCR technology with CO₂ recirculation for buildings;

FIG. 5: CCR technology with CO₂ recirculation for cars.

### DETAILED DISCLOSURE OF THE FIGURES

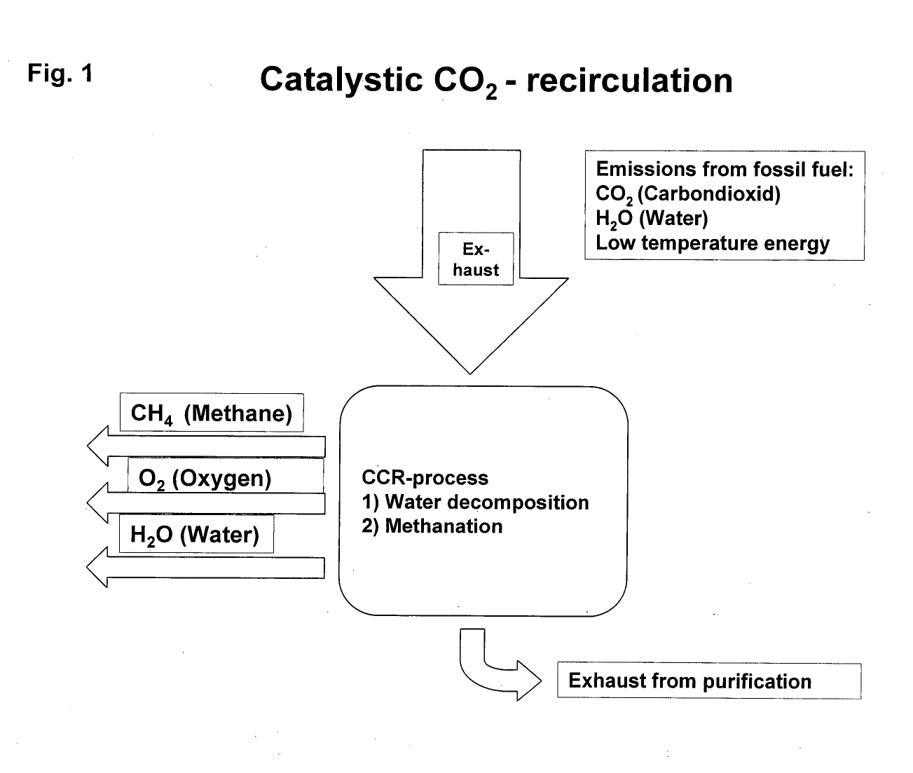

FIG. 1. The figure shows schematically the CCR technology in any power-producing plant based on fossil fuel. The water in the exhaust gas is split into hydrogen and oxygen while the hydrogen reacts with CO₂ in the exhaust gas into methane. The methane and oxygen may either be re-circulated or be used as a raw material in other processes.

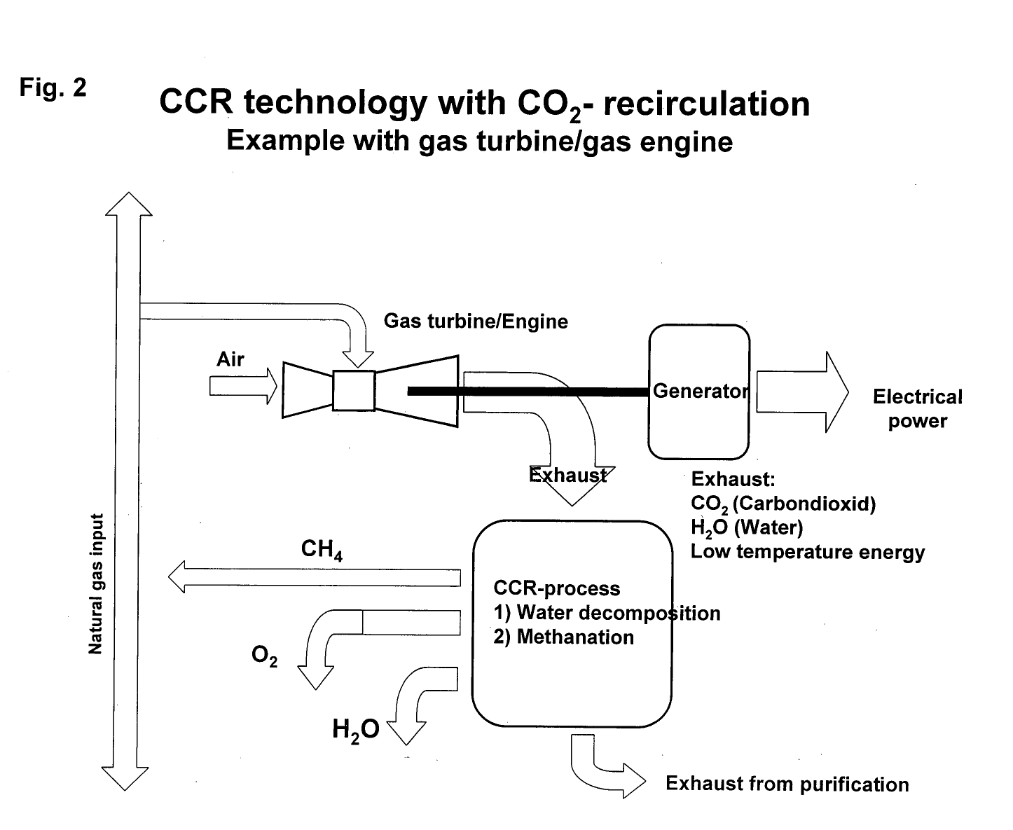

FIG. 2. The figure shows schematically the same as FIG. 1, but with the recirculation of the formed methane for a gas turbine/engine. The oxygen and the water may also be re-circulated or be used in other processes.

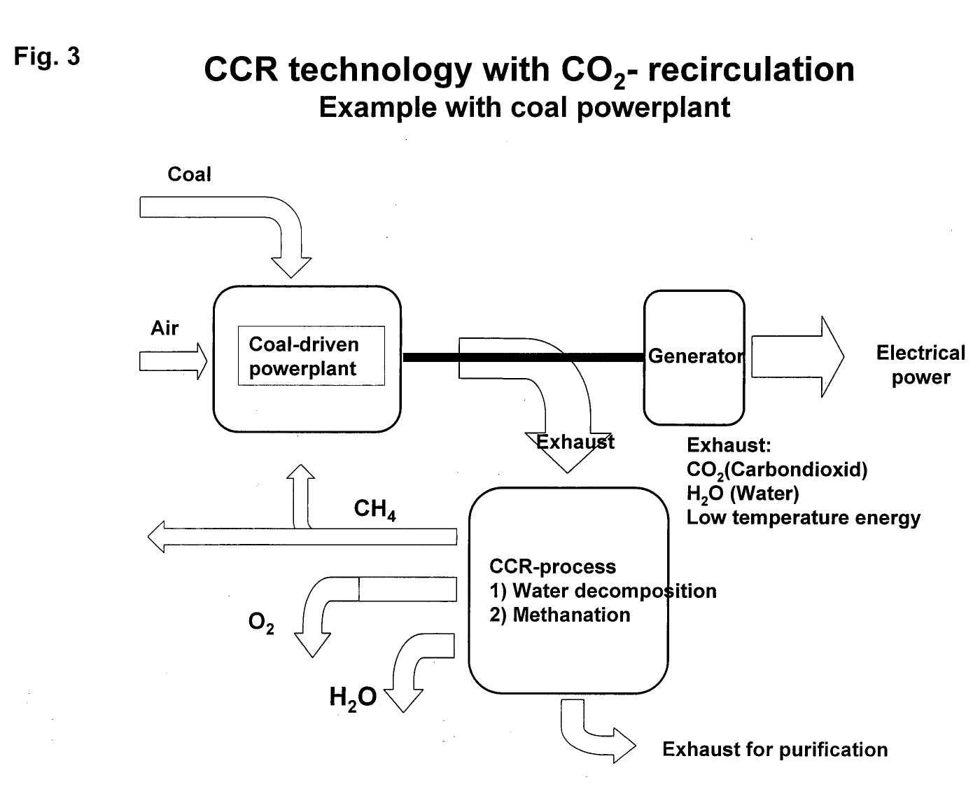

FIG. 3. Shows the same as FIG. 2, but for a coal-fueled power plant wherein parts of the produced methane may be re-circulated.

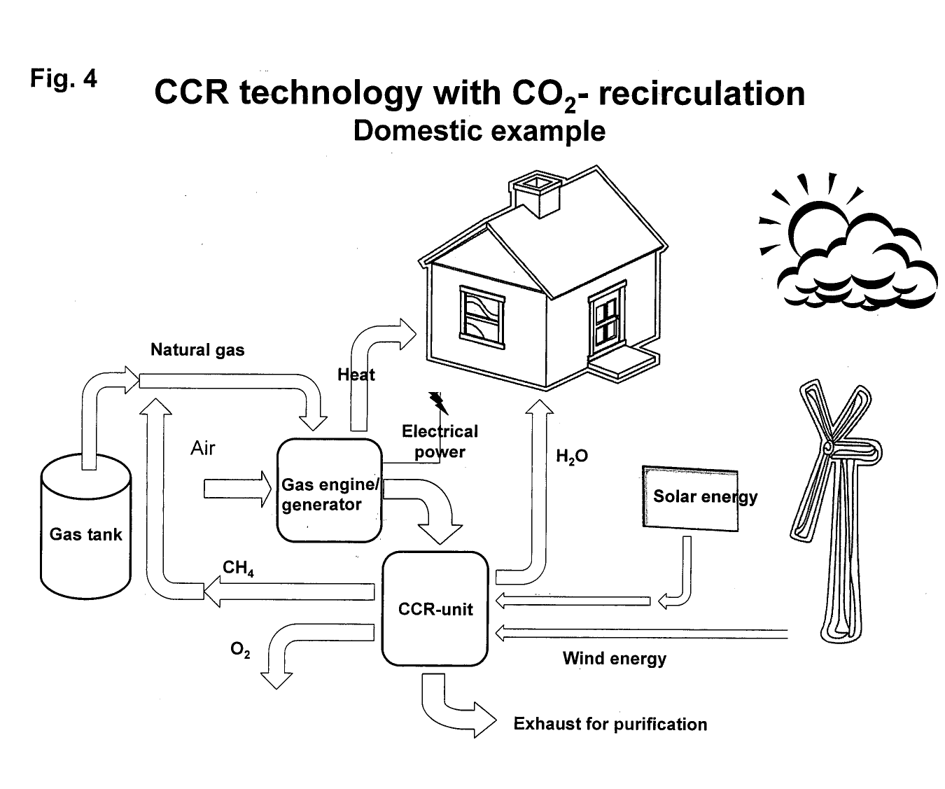

FIG. 4. Shows an arrangement for a house.

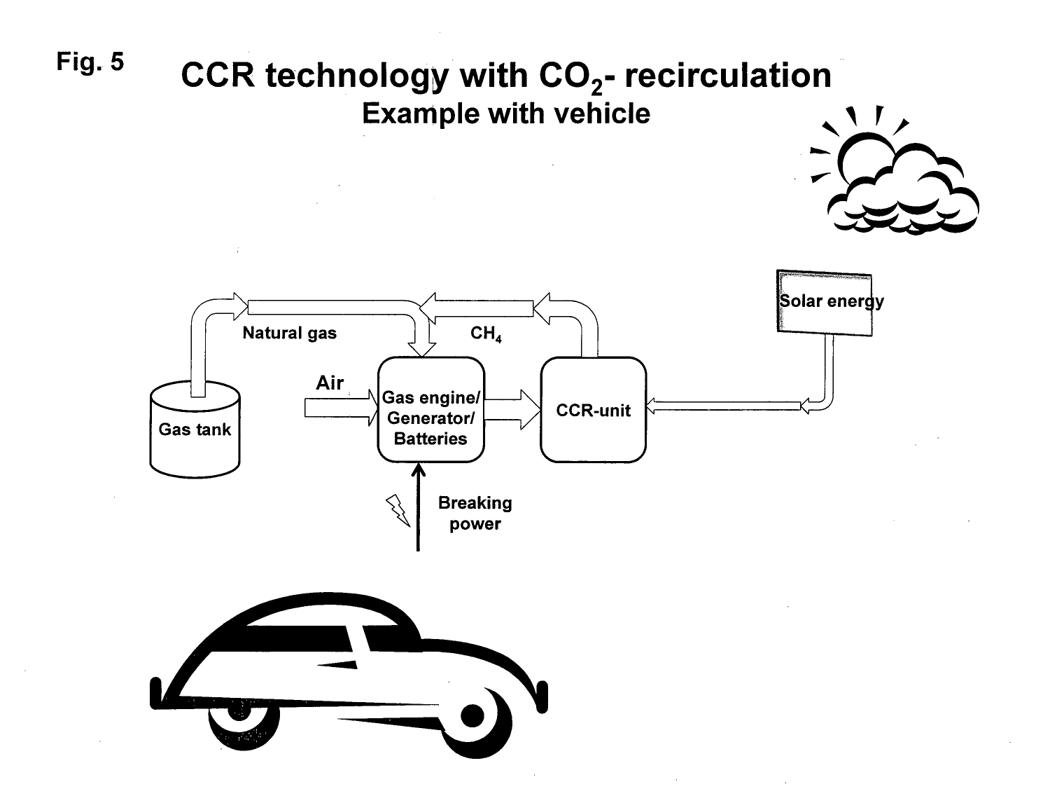

FIG. 5. Shows an arrangement that may be used for a car.

CO₂ may be compressed and stored in a suitable way.

Example 1 of Thermo Chemical Water Dissociation Combined with Methanation.

A thermo chemical cycle for H₂ and O₂ production based on CeO₂/Ce₂O₃ oxides may be used in a combined process with water dissociation and CO₂ methanation. It consists of three chemical steps:

(1) reduction 2CeO₂→Ce₂O₃+0.5O₂
(2) hydrolysis Ce₂O₃+H₂O→2CeO₂+H₂ and
(3) methanation CO₂+4H₂→2H₂O

The hydrogen recovery step (water dissociation with Ce(III) oxide) is performed in a solid bed reactor and the reaction is complete with rapid kinetics in the temperature range 300-500° C. The reformed Ce(IV) oxide is then recycled in the first step. In this process the water is the only material supply and heat is the sole energy addition. The only exit materials are hydrogen and oxygen and these two gases are obtained in different steps to avoid a temperature energy consuming gas phase separation. Furthermore, the oxygen may be used as a source for oxygen in the combustion reaction with water and CO₂ as inert gases instead of air. The hydrogen will be used together with the CO₂-containing exhaust gas and reacted over a methanation catalyst for providing methane and water.

Example 2 of Thermo Chemical Water Dissociation Combined with Methanation.

Large amounts of hydrogen or oxygen may be produced at moderate temperatures (300-900° C.) if a mixed conducting (i.e. electron and ion conducting) membrane is used to remove either oxygen or hydrogen since it is produced by using membranes consisting of an oxygen ion conductor, Gd-doped CeO₂ (CGO) and an electron conductor, Ni, Cu or similar. The water vapor in the gas will react over the membrane separating oxygen from the exhaust gas and leaving the hydrogen in the exhaust gas. The exhaust gas is passed over a methanation catalyst wherein CO₂ reacts with hydrogen for providing methane and water. Furthermore, the oxygen may be used as a source for oxygen in the combustion chamber with water and CO₂ as the inert gases instead of air.

In all examples the excess of heat from the Sabatier reaction (methanation of CO₂ and hydrogen for providing methane and water) will be used either to heat the water-dissociating reaction or for creating any type of energy.

Example of Photochemical Water Dissociation Combined with Methanation.

Water dissociation may be performed by using sunlight as an energy source. The light intensity of the light spectrum from the sun may be 100 mW/cm². Both sides of the photo anode will be illuminated. The cathode will be TiO₂ nano tubular matrix coated with Pt nano particles. 1 M KOH may be used a an electrolyte. Water dissociation will be performed under extreme control conditions by using either a three-divided electrode (with Ag/AgCl as reference electrode) or a two-electrode configuration. In any case the cathode will be in a separate glass-sintered room easing separate removal of hydrogen being made on the cathode surface. The photo generated hydrogen will be fed directly through the methanation system whereas the pure oxygen being created will be used as a combustion gas or by external sources.

A Sabatier-reactor consisting of TiO₂ nano tubular channels coated with a methanation catalyst will methane the hydrogen being formed and the CO₂-gas in the exhaust gas. The catalyst-coated TiO₂ nano tubular template will be rolled up for forming compact layered reaction channels and located inside a specially formed Sabatier reactor. The reactor will be made of acid-resistant steel and have devices for entry and exit of gas. The reactor will have a possibility for external cooling to control the temperature. When the Sabatier-reaction has been initiated the temperature will, on account of exothermal heat production, increase past the set temperature and may sinter the catalyst. Extern cooling of the reactor will aid in controlling the temperature at the set point. Tests will be conducted at 20-350° C.

In all examples air or reintroduced CO₂, water and oxygen can be used as a combustion gas.

1. Process for reducing CO2-emission from the combustion of organic materials with oxygen-containing gas forming carbon monoxide (CO) and carbon dioxide (CO2) as well as water (H2O) wherein at least the formed carbon monoxide and carbon dioxide and water produced through the combustion is passed into a two-step catalytic gas reactor that in its first step includes a catalyst forming hydrogen and oxygen by dissociating water and in its second step includes a catalyst forming methane from reactions wherein CO, CO2 and hydrogen participate according to a methanation scheme as follows:
CO+H2O=CO2+H2 1.
CO+3H2=CH4+H2O 2.
CO2+4H2=CH4+2H2O 3.
H2O=H2+½O2 5,
wherein the flue gas, before the dissociation of water into hydrogen and oxygen, is recycled to the combustion and used as an inert gas by parts or all of the formed oxygen at the dissociation of water being passed back to the combustion of the organic material, wherein energy released from the methane-forming reactions 2 and 3 as well as energy included in the flue gas and/or from sun and wind energy is used to dissociate water into hydrogen and oxygen through reaction 5 over a suitable catalyst material.

2. Process according to claim 1,
wherein at least parts of the hydrogen being formed at the reaction between carbon monoxide and water is returned to the second step of the reactor for the forming of methane.

3. Process according to claim 1,
wherein it is performed without any addition of nitrogen-containing gas (such as air) for avoiding the forming of nitrogen oxides.

4. Process according to claim 1,
wherein parts or all of the formed methane is used as starting material for other processes.

5. Process according to claim 1,
wherein the formed oxygen is used as a starting material for other processes.

6. Process according to claim 1,
wherein the formed CO2 in the exhaust gas being emitted is caught and stored.

7. Process according to claim 1,
wherein the formed CO2 in the exhaust gas being emitted is caught and used in other connections.
